(19) United States
(12) Patent Application Publication
(10) Pub. No.: US 2008/0108548 A1
(43) Pub. Date: May 8, 2008

(54) Food Composition For Rapidly Attenuating Inflammatory Responses

(75) Inventors: Michael Derek Philip Luyer, Maastricht (NL); Adrianus Johannes Maria Vriesema, Houten (NL); Willem Andries Buurman, Eijsden (NL); Johannes Wilhelmus Maria Greve, Maastricht (NL); Robert Johan Joseph Hageman, Wageningen (NL); Zandrie Hafman, Bennekom (NL)

Correspondence Address: FOLEY AND LARDNER LLP;SUITE 500, 3000 K STREET NW, WASHINGTON, DC, 20007, US

(73) Assignee: N.V. Nutricia

(21) Appl. No.: 11/719,193

(22) PCT Filed: Nov. 14, 2005
(86) PCT No.: PCT/NL05/50040

§ 371 Date: Jun. 29, 2007

(30) Foreign Application Priority Data
Nov. 12, 2004 (EP) 04078112.2

(51) Int. Cl. A61K 38/02; A61P 29/00

(52) U.S. Cl. 514/2

(57) ABSTRACT

The invention pertains to the use of a lipid fraction and/or a protein fraction for the manufacture of a composition for causing an immediate attenuation of the inflammatory response. The lipid and/or protein stimulate the parasympathetic nervous system centrally or peripherically via the gastrointestinal tract leading to rapid attenuation of the inflammatory response via stimulation of nicotinic receptors by vagal efferents. The lipid fraction preferably contains 6-50 wt. % of phospho-lipids and the protein fraction preferably comprises intact whey or casein or hydrolysed soy protein. Additionally, components such as mono-sodium glutamate or betaine are included to optimise vagal stimulation.

### FIELD OF THE INVENTION

The invention pertains to methods and compositions for mitigating or preventing acute inflammatory responses and exacerbations of existing chronic inflammation e.g. in surgery, trauma, injury and burns and major chronic diseases with exacerbations.

### BACKGROUND

The body of mammals may respond to stressors with an inflammatory reaction. Stressors can be of infectious or non-infectious origin. Examples of stressors are microorganisms (viral, bacterial, fungal, or parasitical) or non-endogenous proteins or peptide-related materials which have invaded a tissue locally, physical traumas including surgery, severe blood loss, extra-corporal circulation or extensive soft tissue damage and multiple fractures, ischemia/reperfusion events, or damage to cells or tissue due to burns, chemotherapy, radiation or intoxication. During the inflammatory reaction many factors play a role. Inflammatory mediators, including acute-phase proteins, interleukins, prostaglandins and leukotrienes, contribute to a concerted number of responses within the body (i.e. blood flow to the affected tissue, repair processes and combat against undesired exogenous components and apoptosis of endogenous cells) in order to obtain an appropriate host defence-reaction, in particular to rapidly and effectively neutralise stressors, obtain rapid wound healing, prevent tissue damage as caused by ischemia/—reperfusion processes or chemo—or radiotherapy and achieve a fast repair of tissue function.

Patients that are subjected to surgery are mostly fasted prior to surgery in order to prevent potential vomiting and aspiration of stomach contents. In addition, patients often experience serious problems in feeding themselves properly in the period directly after surgery or trauma. Statistics demonstrate that many hospitalised patients suffer from complications. Such complications vary from delayed wound healing, swelling and pain, bad recovery of function of the stressed tissue, local infections, and a compromised immune function, and may in some instances even result in systemic complications like sepsis and multiple organ dysfunction, which are aggravated by decreased nutritional intake.

It is a purpose of the invention to provide nutritional support to patients, who have been or will be exposed to such stressors and are at risk for or are already experiencing complications as a result of these stressors. The provision of nutritional support is practical to apply and prevents in particular the development of a (too) strong or dysregulated inflammatory response during and following the presence of the stressor and the subsequent healing process by a rapid mode of action. When the inflammatory reaction has already started, it prevents a further increase of the inflammatory reaction.

Hence, the problem to be solved by the invention was to provide a pharmaceutical, and preferably a nutritional composition capable of rapidly averting short-time inflammatory effects, in particular associated with exacerbations of chronic inflammatory diseases or with major medical interventions such as surgery, radiotherapy, chemotherapy, or with acute infections, ischemic effects, burns and the like. During several chronic inflammatory diseases, like inflammatory bowel disease, and chronic respiratory diseases, like Chronic Obstructive Pulmonary Diseases (COPD) and asthma, patients may experience periods in which the symptoms of the disease women in a relatively short period. This process is called an exacerbation of the disease. In most cases a patient will, optionally after medical interference, after some time recover from this exacerbation period.

WO 01/89526 (North Shore) describes methods of reducing inflammation caused by pro-inflammatory cytokines or an inflammatory cytokine cascade, in which macrophages are treated with a cholinergic agonist such as acetylcholine, nicotine, muscarine and the like. Stimulation of the efferent parts of the Nervus Vagus, which distribute electric pulses from the central nervous system to the peripheral tissues is suggested as a treatment mechanism. The conditions to be treated include various inflammatory diseases and especially endotoxic shock.

WO 03/009704 (Nutricia) discloses the use of a lipid composition containing phospholipids, triglycerides and cholesterol in a ratio of 3-90:3-80:1 for the treatment of sepsis, which may be associated with major surgery, critical illness, inflammatory bowel disease etc., caused by bacteria.

WO 04/068969 (Nutricia) similarly discloses the use and method of preparation of a lipid composition containing phospholipids and triglycerides in a ratio of greater than 1, without cholesterol, for the treatment of sepsis and associated conditions.

EP-B 1041896 (Nutricia) discloses fat compositions comprising γ-linolenic acid, stearidonic acid and eicosapentaenoic acid (2:1:2) and phospholipids for the treatment of chronic inflammatory diseases, lipid metabolism disorders and weakened immune functions.

EP 0189160 (Abbott Laboratories) discloses a nutritional formula comprising 45-60 percent of the total amount of energy provided by the formula (=en/%) fat, 0-30 en % carbohydrate, 8-25 en % protein, and a micronised calcium salt, wherein the fat fraction can comprise 2.5-50 weight percent emulsifier. The protein can be caseinate salts and the emulsifier lecithin and mono- or diglycerides. This formula is claimed to improve respiratory insufficiency. The document is silent about a physiological role of the emulsifier in the body and about a beneficial effect of the complete product in rapidly weakening the inflammatory response as caused by a stressor.

EP 1090636 (INRA) discloses a composition for use in the treatment of sepsis or inflammatory shock, which comprises more than 35 en % lipid. The lipid fraction comprises 25-70 wt % MCT oil. Less than 15 wt % saturated fatty acids excluding MCT and the n-6/n-3 ratio is about 2-7:1.

U.S. Pat. No. 5,434,183 (Pharmacia) discloses a pharmaceutical composition which comprises phospholipids of marine and/or synthetic origin containing omega-3 fatty acids in an amount of at least 30% (w/w). The composition is claimed to be useful for treating a patient in need of anti-inflammatory and/or immunosuppressive effects, like those suffering from rheumatoid arthritis or sepsis. The composition may comprise conventional carriers and diluents and may be an emulsion and comprise oils, monoglycerides of fatty acids, components for adjusting isotonic properties, anti-oxidants and components for adjusting stability, such as amino acids and carbohydrates and further bioactive components.

### DESCRIPTION OF THE INVENTION

The invention provides a method for preventing and treating acute inflammatory conditions and exacerbations of chronic inflammatory diseases. Such conditions require immediate effect of an intervention, wherein the effect of the intervention consists in attenuation of the inflammatory response. The immediate response means that the attenuation starts within 30 minutes, preferably within 15 minutes, and lasts for several hours, in particular for 1-3 hours after the intervention has stopped.

According to the method of the invention, a nutritional composition comprising a protein fraction and/or a lipid fraction is administer which fraction is capable—probably through induction of the release of active intestinal components and binding thereof to receptors of nerve cells—of activating the nerve cells, in such a manner that efferent vagus nerve activity is stimulated, resulting in inhibition of the inflammatory cascade via nicotinic receptors on inflammatory cells.

Indeed, it was found by the inventors that the nutritional compositions containing specific lipid and/or protein fractions rapidly stimulate the parasympathetic nervous system (centrally or peripherically) contributing to a rapid attenuation of the inflammatory response via stimulation of nicotinic receptors on macrophages by vagal efferents. Vagal efferents are those parts of the nervus vagus which transport impulses from the central nervous system to the periphery and vagal afferents transport impulses from the periphery to the central nervous system. Vagotomy blunted the inhibitory effect of the specific lipid and/or protein fractions on hemorrhagic shock-induced circulating tumour necrosis factor (TNF)-alpha, interleukin (IL)-6, endotoxin and intestinal permeability for HRP in rats. In addition, antagonists for CCK-receptors and inhibition of nicotinic receptors increased the pro-inflammatory response and deteriorated intestinal barrier function caused by hemorrhagic shock in rats, despite the treatment with the specific lipid and/or protein fraction.

The invention also pertains to a pharmaceutical or nutritional composition for inducing an immediate attenuation of inflammatory response, comprising a lipid fraction and/or a protein fraction as described below, optionally together with further components.

### Lipid Fraction

The lipid fraction that can be used according to the invention preferably comprises at least 6 wt. % of phospholipids, up to 50 wt. %, based on the total lipid fraction. The preferred phospholipid content is 8-50 wt. % of the lipid fraction, especially 10-35 wt. %, most preferably 12-30 wt. %. The phospholipids may comprise any phosphoglycerol derivative having at least one long-chain (≧C16) fatty acyl residue, including diacyl phospholipids) and monoacyl (lysophospholipids) derivatives, such as phosphatidyl-ethanolamine (PE), phosphatidylcholine (PC), phosphatidylserine (PS), phosphatidyl-inositol (PI), phosphatidylglycerol (PG), phosphatidic acid (PA) etc. and their lyso analogues. Preferably PE and PC are present for at least 3 wt. %, most preferably at least 6 wt. % of the lipid fraction and/or at least 30 wt. % of the phospholipids, especially in a ratio of PC/PE between 10:1 and 1:1, more in particular between 5:1 and 1.2:1. Preferably, the level of PS is below 10 wt. %, especially below 2 wt % of the total phospholipids. The nature of the fatty acids in the phospholipids is thought not to be essential to the observed effect. Typically the fatty acids in the phospholipids comprise less than 90 wt % and preferably less than 80 wt. % linoleic acid, and the amount of ω-3 polyunsaturated fatty acids, in particular eicosapentaenoic (timnodonic) acid, docosa-hexaenoic (cervonic) acid, is less than 30 weight percent and preferably 2-26 wt %.

In addition to the phospholipids, the lipid fraction comprises a glyceride fraction. This may contain mono-, di- and tri-glycerides. Preferably, in order to facilitate rapid digestion, part of the glyceride fraction consists of mono and/or diglycerides of fatty acids. The mono- and diglycerides were also found to assist in administering relatively large amounts of lipids, without excessively raising the caloric content of the composition. Preferably the sum of mono- and diglycerides is between 2 and 50 wt. % of the lipid fraction, more preferably between 4 and 20 wt %. Taken individually, the diglycerides preferably account for 140 wt. %, more preferably 2-20 wt. % of the lipid fraction and the monoglycerides for 0-30, more preferably 1-15 wt. %. The remainder of the lipid fraction may consist of triglycerides. In particular, the triglyceride content of the lipid fraction may be between 20 and 90 wt %, especially between 30 and 60 wt %.

The fatty acid composition of the lipid fraction preferably largely, i.e. for more than 75 wt. %, consists of fatty acids which have a chain length of 16 or 18 carbon atoms. Preferably the C18 content is 45-95 wt. %, more preferably 55-95 and most preferably 70-94 wt. %. Among the C18 fatty acids, preferably 10-50 especially between 15 and 50 wt % consists of polyunsaturated fatty acids, especially linoleic and α-linolenic acid. In view of their reduced stability (off-flavours), the level of γ-linoenic acid (ω-6 octadecatrienoic acid, GLA) and stearidonic acid (ω-3 octadecatetraenoic acid, SA) is relatively low, i.e. preferably less than 6 wt. %, especially less than 2 wt. %, GLA and SA taken together, of the fatty acid composition.

The remainder of the fatty acid residues may be formed by medium-chain fatty acids, having a chain length of 8, 10 or 12 carbon atoms, preferably in an amount of 0-20 wt %, more preferably 0-6 wt. %, and especially less than 3 wt. %, and myristic acid (C14:0). The amount of the saturated fatty acids myristic acid, palmitic acid and stearic acid is typically 1-30 wt. %, preferably 5-25 and more preferably 16-22 wt. %. The amount of long-chain fatty acids having a chain length of 20 or more is 0-12 wt. %, especially 1-6 wt. %. The long-chain fatty acids, if present, may comprise timnodonic acid (ω-3 eicosapentaenoic acid, EPA), clupanodonic acid (ω-3 docosapentaenoic acid) and cervonic acid (ω-3 docosahexaenoic acid, DHA), even though their content should not be too high in view of their limited stability. The EPA content with sect to the total of EPA, GLA and SA is preferably more than 50 wt. %, and the SA content with respect to the same total of EPA, GLA and SA is preferably smaller than 15 wt. %, and preferably less than 10, more preferably less than 6 wt/o. Cholesterol should preferably not be included at a level exceeding 0.5 wt. % of the lipid fraction, and is preferably not present.

Although the lipid fraction may be the only energy carrier of the composition to be used according to the invention, it is preferred that the composition also contains carbohydrates and/or proteins, preferably at least proteins. The energy contribution of the lipid fraction to that of the total composition is preferably 42-90 end, more preferably 44-75 en %, most preferably 45-60 en %. Thus, the invention also pertains to the use of a food composition containing proteins, carbohydrates and lipids, wherein the fats comprise 42-90%, in particular 45-60% of the energy content of the composition, for preparing a medicinal food for stimulating vagal afferents of the parasympathetic nervous system and/or for rapidly attenuating the inflammatory response via stimulation of nicotinic receptors by vagal efferents.

### Protein Fraction

The protein fraction to be used according to the invention is preferably selected from milk proteins and soy proteins. The milk proteins are preferably intact proteins. The milk proteins may exclusively be casein or exclusively whey protein, or a mixture thereof. In a mixture, the weight ratio of casein to whey protein may e.g. be between 6:1 and 1:6. It is preferred that at least 18 wt. %, especially 40-100, in particular 55-90 wt % of the protein fraction consists of whey protein. In a preferred embodiment, the whey proteins comprise a relatively high proportion of alpha-lactalbumin, preferably at least 10 wt. %, especially between 20 and 90 wt. %, and more preferentially between 36 and 70 wt. % of α-lactalbumim, The weight ratio of α-lactalbumin to β-globulin in the whey proteins is in the range 1-100:1, preferably 5-20:1, more preferably 6-16:1. The content of α-lactalbumin can be increased by using methods well known in the art, e.g. by separation of lipid and casein fraction and using chromatographic methods to separate the different whey proteins. Pure α-lactalbumin and α-lactalbumin-enriched whey extracts are commercially available. Preferably, the α-lactalbumin content of the protein fraction is at least 5 wt %, more preferably between 10 and 60 wt %.

Alternatively, at least 40 wt % or even the majority or the entire protein fraction may be formed by hydrolysed soy protein. The soy protein can be hydrolysed by pepsin, trypsin, chymotrypsin or other commercially available proteases or mixtures thereof. More preferably the soy protein has been hydrolysed by subjecting it to at least a treatment with pepsin. The degree of hydrolysis is preferably such that at least 50 wt % of the hydrolysate has a molecular weight of less than 10 kDa or at least 50 wt % consists of peptides of less than 90 amino acids. The presence of these particular proteins in the product is found to increase the duration of the action of the lipid fraction on the effect of the nervus vagus on host response to stressors and therewith decrease the amount of lipid or the frequency of dosing of the lipid enriched product.

In addition to the milk proteins and/or the hydrolysed soy protein, specific amino acids may advantageously be present in the protein fraction. Prefer amino acids include glutamate, glutamine, phenylalanine and tryptophan. These amino acids may be present as such, i.e. as isolated amino acids, but preferably as relatively small peptides selected from specific proteins that are known to be rich sources of these amino acids. Typical chain lengths are 2-90, preferably 2-40 and more preferably 2-20. An example of a suitable glutamate source is monosodium, monopotassium or magnesium or calcium glutamate or mixtures thereof. The amount of glutamate in the product can be increased by adding more than 0.2 g glutamate source per 100 g protein, and preferably 0.5-3 g per 100 g protein as salt or as peptide material, which will typically lead to a glutamate content of more than 16 wt % based on protein and preferably 17-20 wt %. Tryptophan levels are typically more than 1.6 wt % and preferably 1.7-3.5, most preferably 1.9-2.8 wt % based on protein. Phenylalanine levels can be increase by adding more than 0.2 wt % of phenylalanine source, preferably 0.5-3 wt % based on protein, resulting in a phenylalanine level of typically 5.7-8 wt % based on protein.

The protein fraction preferably does not comprise effective amounts of intact undenatured IgY immunoglobuluis. Typically the concentration of IgY is less than 200 μg per daily dose or less than 200 μg per liter product, or less than 200 μg per 100 g protein, and preferably less than 100 μg per daily dose or per liter product or per 100 g protein. On the other hand it is beneficial to include 10-50 wt % of the intact whey protein as glycomacropeptide and preferably 20-46 wt %. The whey protein therefore preferably originates from sweet whey.

Although the protein fraction may be the only energy carrier of the composition to be used according to the invention, it is preferred that the composition also contains carbohydrates or lipids, preferably at least lipids. The energy contribution of the protein fraction to the total composition is preferably 6-50 en %, more preferably 10-40 or 15-40 or even 17-35 en %, and most preferably 20-30 en % or alternatively 10-25 en %.

### Further Components

The nutritional composition of the invention can further comprise a digestible carbohydrate fraction. The carbohydrate fraction contributes between 0 (no digestible carbohydrates) up to 50 energy % of the composition, preferably 4-40% or 10-40 en %, most preferably 20-36 en %. The carbohydrates may comprise maltodextrins, glucose syrups, hydrolysed starches, soluble starches, monosaccharides like glucose, fructose, galactose, mannose, etc. and disaccharides like sucrose and lactose. No specific mixture is preferred, on the condition that the osmotic value of the final product is below 400 mOsmol/liter, in particular in the range of 250-380 mOsm/tl. The product does not comprise lactose if it is intended to be consumed by persons that suffer from lactose intolerance. The ratio of carbohydrates to lipids is preferably between 1:0.81 and 1:5, more preferably between 1:1.0 and 1:4, on an energy basis. These requirements for the amounts of protein, lipid and carbohydrate in the product result in a relative contribution of the caloric content of protein:lipid:carbohydrate=0.45-2:0.8-5:1, preferably 0.6-1.8:0.94.5:1 and more preferably 1-1.6:1-4:1.

The composition of the invention may further contain other nutritional components, such as vitamins and minerals, e.g. in an amount of between 0.2 and 1.0 tires the recommended dosages of the vitamins, minerals etc. It is preferred to include betaine rather than choline because of its organoleptic properties. As source of betaine any food-grade ingredient can be used that releases the amount of betaine as desired according the invention. Examples include synthetic betaine inner salt, either anhydrous or hydrated forms, its salts, e.g. mixtures of HCl salts and other acids, like carbonic, adipic acid, suberic acid, sebacic acid, sulphuric acid, acetic acid, citric acid, malic acid, and acidic amino acids, like aspartic acid and glutamic acid in any hydrated form. Also extracts of plant or animal material like extracts from sugar beet and mixtures of betaine and guanidino acetate can be used. However, it is preferred to use either the inner salt of betaine or salts with organic acids like citric acid and malic acid or amino acids. In particular the salt of aspartic acid and betaine is preferred. Taken alone, the amount of betaine is preferably from 0.2 to 20 wt. %, preferably 0.4-10 wt. %, most preferably 0.5-5 wt. % calculated on dry matter. If combined, the betainetcholine weight ratio is preferably at least above 1, more preferably between 1.5 and 9.

Carnitine and inositol are other ingredients that are advantageously present in the composition. Suitable sources of carnitine are the food grade ingredients of the D- or L-form or mixtures thereof. Carnitine sources are preferably alkanoyl-cartines like propionyl, acetyl, isobutyryl or isovaleroyl, or isopropyl or isovaleryl caritine. Suitable amounts are 0.1-2 g per litre product. Inositol can be food grade qualities of myo-inositol Suitable amounts are 10-1000 mg per litre product

It is also preferred that the composition contains dietary fibre (=poorly digestible or non-digestible carbohydrates). Preferred amounts are between 0.5 and 5 wt %, based on total dry weight. The dietary fibre may comprise oligosaccharides such as oligo-fructoses including inulin, galacto-oligosaccharides, arabino-oligosaccharides and xylo-oligosaccharides and the like and combinations thereof soluble non-starch poly-saccharides, such as galactan gums, (galacto/gluco)manan gums, xyloglucan gums, beta-glucans, pectins, etc. and non-soluble polysaccharides, such as cellulose, hemi-cellulose, and resistant starch In particular soluble fibre ingredients are desirable, e.g. between 0.5 and 4 wt. %. It is beneficial to combine the prebiotics with probiotics like lactobacilli, in particular Lactobacillus rhamnosus, bifidobacterial species and propioni-bacteriae to increase the effect on the immune system. In case the product has a liquid form, the probiotics are preferably either added to the liquid product shortly prior to consumption or are administered separately within the same meal. In case the product is a dry product, having a moisture content below 4 wt %, and preferably below 2 wt %, the probiotic ingredient can consist of freeze-dried material which comprises 10⁸ to 10¹⁰ viable or dead bacteria per gram ingredient or at least 0.5 g bacteria fragments per gram ingredient.

The composition may further comprise a carbonate fraction. Suitable ingredients include carbonate salts and/or bicarbonate salts, e.g. sodium, potassium, magnesium, ammonium and/or zinc salts. The final pH of a liquid product, as well as the mineral composition of the final product will determine relative amounts in solution. The amount of added carbonate fraction during manufacture of the product is preferably 0.1-2 wt % of the dry mass of the formula in order to stimulate digestion of the product. The final pH of the product in solution is between 4 and 8, preferably between 5 and 7. It is further preferred that the composition comprises 1-10 wt % and more preferably 1.5-6 wt % of cacao mass based on dry matter. This contributes to the levels of macroingredients because suitable ingredients for cacao provide 18-22 wt % protein, 20-30 wt % lipids, 8-12 wt % digestible carbohydrates and 20-34 wt % fibre. Inclusion improves palatability and increases the effect of the preparation.

The composition when used in humans is preferably a liquid composition suitable for oral administration (drink). The energy density of the composition for complete nutrition of the patient may be between 0.75 and 1.75 kcal/ml (3.14-7.32 kJ/ml), preferably between 0.96 and 1.44 kcal/ml (4.0-6.0 kJ/ml). For example, 100 ml of the liquid composition may contain 5-10 g of proteins, 4-10 g of lipids, 4-14 g of digestible carbohydrates and 60-5000 mg (preferably 70-3500 mg, more preferably 100-2500 or even 200-2000 mg of betaine (or choline). The composition may comprise trace elements, vitamins and minerals as conventional in liquid nutrition. No special measures need to be taken like encapsulation or micronising salts for achieving the effect of the invention.

The composition according the invention can be a supplement and or a complete formula and is meant to be used for oral use. It can be applied by drinking or by tube feeding. When the product is a supplement to be used for pharmaceutical purposes, it will be used in smaller quantities and therefore be more concentrated. Typically it will provide between 1 and 4 kcal per millilitre and preferably 1.4-3 kcal/ml. Nutritional supplements may optionally include, apart from the protein and lipid fraction according the invention, nutritional components that are beneficial to the specific patient. These can be drugs but also nutrients that are of interest to treat deficiencies on specific minerals, trace elements and vitamins and a carbonate fraction.

When used in animal nutrition the product will take the form of a liquid, a slurry or a dry product, the latter preferably being a granulate or a pellet Ingredients that are used in the preparation of animal nutrition differ from those that are used in the preparation of human nutrition but are known in the art and include soy, dairy products, fish meal, feather meal, blood meal, eggs, pure amino acids, bone meal, calcium phosphates, lime stone, minerals, vitamins, trace elements, corn, peas, cereals, like wheat, barley, triticale, oats (flakes), rapeseed meal, lupine, corn germ, sunflower, sugar beet pulp, molasses, cacao mass, gelatinised starches, potato, etc. or fractions thereof. Typical nutrition for piglets comprises per 100 g dry matter 1.3-1.9 MJ, 16-22 g crude protein of which at least 6.5 wt % is lysine, 0.5-5 g crude fibre, but according to the invention comply with the relative composition of protein, lipids, digestible carbohydrates and other features, as defined in the claims, by mixing the ingredients as mentioned. Liquid formulae for piglets will typically comprise per 100 ml 4.8-5.8 g crude protein, 4.8-5.5 g lactose apart from lipids, ash 0.6-1.0 g and vitamins and optionally other components. The lipid fraction in piglet nutrition will typically comprise, based on fatty acid content, 45-85 wt % and preferably 55-75 wt % fatty acids of chain length 18 and will in the particular case of piglet nutrition comprise 50-80, preferably 55-75 wt % oleic acid. In this case the amount of long chain poly-unsaturated fatty acids is less than 25 wt % and preferably 5-18 wt %.

### Use of the Composition

The method and composition of the invention can be used for attenuating the inflammatory response in acute inflammatory situations as caused by one or more stressors like surgery, radiotherapy, chemotherapy, acute (bacterial) infections, ulcerations, trauma, injury, severe blood loss, blood transfusions, ischemic events, heart failure, burns or depression of immune status, or during periods of exacerbation of chronic inflammatory diseases.

In the method of the invention, the condition to be treated or prevented can in particular be associated with diseases or disorders selected from the group consisting of appendicitis, peritonitis, pancreatitis, ulcerative, pseudomembranous, acute and ischemic colitis, diverticulitis, epiglottitis, Alzheimer's disease, cholangitis, cholecystitis, Crohn's disease, enteritis, allergy, immune complex disease, organ ischemia, reperfusion injury, organ necrosis, hay fever, sepsis, septicaemia, Acute respiratory distress syndrome (ARDS), Acute renal failure (ARF), (multiple) organ failure, Systemic inflammatory response syndrome (SIRS), Compensatory anti-inflammatory response syndrome (CARS), Necrotizing enterocolitis (NEC), diabetic foot ulcers, endotoxic shock, cachexia, hypepyrexia, eosinophilic granuloma, granulomatosis, sarcoidosis, septic abortion, epididymitis, vaginitis, prostatitis, urethritis, rhinitis, cystic fibrosis, pneumonitis, alveolitis, pharyngitis, pleurisy, sinusitis, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, coeliac disease, congestive heart failure, meningitis, encephalitis, multiple sclerosis, cerebral infarction, cerebral embolism, Guillain-Barré syndrome, neuritis, neuralgia, spinal cord injury, paralysis, uveitis, arthritidis, arthralgia's, osteomyelitis, fasciitis, Paget's disease, gout, periodontal disease, rheumatoid arthritis, synovitis, myasthenia gravis, thyroiditis, systemic lupus erythematosus, Goodpasture's syndrome, Behcet's syndrome, allograft rejection, graft-versus-host disease, Type I diabetes, ankylosing spondylitis, Type II diabetes, ankylosing spondylitis, Berger's disease, and Hodgdkin's disease.

In particular, the condition is associated with appendicitis, peptic, gastric and duodenal ulcers peritonitis, pancreatitis, ulcerative, pseudomembranous, acute and ischemic colitis, hepatitis, Crohn's disease, allergy, anaphylactic shock, organ ischemia, reperfusion injury, organ necrosis, hay fever, sepsis, septicaemia, endotoxic shock, cachexia, septic abortion, disseminated bacteremia, burns, ARF, SIRS, CARS, ARDS, (multiple) organ failure, NEC, coeliac disease, congestive heart failure, cerebral infarction, cerebral embolism, spinal cord injury, paralysis, rheumatism, rheumatoid arthritis, allograft rejection and/or graft-versus host disease.

Other conditions for which intervention can be indicated include stay in the intensive care unit of a hospital like patients that already are critically ill or those patients that experience a risk of getting critically ill. Examples of the latter category are patients that are subjected to types of surgery which cause a relatively high risk for complications, like those to the gastrointestinal tract, long-lasting surgery or surgery to critical organs, like pancreas or liver, but also surgery to patients that are malnourished or are in a fasted state or are suffering from hypocholesterolemia, or patients suffering from cystic fibrosis. In particular the product can be administered pre-operatively, i.e. between 1 day and 5 min, in particular between 8 hours and 30 min, or even between 4 hours and 15 min, especially between 90 and 15 min, before option, but also optionally in those cases wherein the condition of the patients worsens or changes in such a way that nutritional intervention is beneficial. Such cases include other physical interventions in the body, such as incisions, transplantation, blood transfusion, as well as anticipated exposure to allergens, pollutants or inhaled irritants. Especially, the method of the invention is useful in preventing ileus, i.e. reduced motility of the digestive tract such as intestinal obstruction causing colic, vomiting, and constipation, that may result from initial inflammation signals, such as those created by surgery or other stressors, like obstructions by gallstones or kidney stones or acute peritoneal contamination.

The method and use of the invention is particularly effective for persons at risk for an acute inflammatory event or exacerbation of chronic inflammatory disease. The latter risk is markedly enhanced in persons that have suffered from inadequate nutritional intake, i.e. patients who are malnourished or fasted or in a catabolic state with increased risk of complications and poor recovery at the time of the nutritional intervention. Criteria for establishing that a patient suffers from malnourishment have been described in the medical literature and include determination of lean body mass, determination of food intake in the recent past and measurement of relevant parameters in blood plasma

When exacerbation of chronic inflammatory diseases such as Crohn's disease, COPD or asthma is signalled, e.g. by symptoms such as increased abdominal pain or diarrhoea in case of Crohn's disease, or increased or altered sputum production in case of COPD, the method of the invention is preferably used within 5 minutes to 1 or more days after the onset of the exacerbation, or within 5 minutes and 1 day before an anticipated repeat of the signal, e.g. in case of anticipated exposure to stress factors such as medical interventions, allergens, irritants, etc. Administration of the food composition containing 42-90% of fat or the fat and/or protein fraction of the invention quickly after the first symptoms of exacerbation helps in preventing aggravation of the disease as a result of the immediate effect of the food on the inflammatory response.

According to the invention, the compositions as disclosed above have been found to be very useful in mitigating and/or preventing acute inflammatory response in mono-gastric or polygastric mammals like pig, cate (calves), dog and cat, especially those inflammatory responses that frequently are observed after weaning and in the case of pigs during the process of transport and slaughtering or other stressful events. The products should preferably be administered several hours to shortly, e.g. 24 hours to 5 minutes, especially between 4 hours and 15 minutes before the stressful event is planned or expected to happen.

### EXAMPLES

### Example 1

### Tube-Feed with the Following Composition

| | |
|---|---|
| Energy | 125 kcal |
| Carbohydrates | 9.375 g (30 en %) |
| Proteins | 6.875 g (22 en %) |
| Casein | 6.875 g |
| (Total) fat content | 6.67 g (48 en %) |
| phospholipids | 1.66 g (25 wt % of total fat) |
| linoleic acid | 0.42 g-1.39 g (3-10 en %) |
| α-linolenic acid | 0.07 g-0.625 g (0.5-4.5 en %) |
| n-6/n-3 ratio | 2:1-6:1 |
| Vitamins and minerals | 100% of RDA's |
| Fibre (current Stresson fibre mix) | 1.5 g |

### Example 2

Liquid for preoperative use, providing 1.2 kcal per ml and comprising per 100 ml: 6.3 g protein (80 wt % intact casein and 20 wt % intact whey protein, 0.2 g monosodium glutamate), 8.0 g lipids (canola oil, 25 wt % phospholipids, fish oil, milk fat), 4.8 g digestible carbohydrates (glucose syrup, maltodextrins, sucrose), 0.9 g betaine, 2 g ash (sodium bicarbonate, potassium chloride, magnesium and calcium salts).

### Example 3

Liquid for use in preventing exacerbations in chronic inflammation (Crohn's Disease, COPD) comprising per 100 ml, 120 kcal and:

5.9 g protein (80 wt % milk protein+20 wt % alpha-lactalbumin enriched whey), 6.8 g lipids (30 wt % egg phospholipids), 8.0 g digestible carbohydrates, 3 g dietary fibre (galacto-oligosaccharides, oats bran, wheat bran), and microingredients (80 mg Na, 135 mg K, 125 mg Cl, 57 mg Ca, 57 mg P, 20 mg Mg, 1.0 mg Fe, 1.0 mg Zn, 0.15 mg Cu, 0.3 mg Mn, 0.1 mg F, 5 μg Mo, 4.3 μg Se, 3.3 μg Cr, 10 μg I, 67 μg RE vit A, 0.5 g vit D, 0.81 mg α-TE, 4 μg vit K, 0.1 mg vit B1, 0.11 vit B2, 2.6 mg NE niacin, 0.4 mg pantothenic acid, 0.13 mg vit B6, 13 μg folic acid, 0.2 μg vit B12, 10 μg biotin, 5 mg vit C, 20 mg choline, 0.2 g betaine) and 2 g cacao mass.

### Example 4

Liquid formula for ICU patient providing per 100 ml 120 kcal and comprising: 6.8 g protein (30 wt % hydrolysed soy protein isolate, 70 wt % milk protein), 6.6 g lipids (5 en % linoleic acid, 3 en % α-linolenic acid), 20 wt % phospholipids), 9.5 g digestible carbohydrates, 0.2 g betaine, 0.2 g dietary fibre, organic acids, minerals, trace elements and vitamins according to nutritional guidelines for clinical nutrition, wherein carbonates are used as salts making carbonate content 0.2 wt % of dry mass.

### Example 5

### Materials and Methods

Using the non-lethal hemorrhagic shock model as described earlier (Luyer et al., Ann. Surg. 239:257-264, 2004; Luyer et al. Shock 21:65-71, 2004) rats were anesthetised, the femoral artery was dissected and cannulated. Mean arterial pressure (MAP) and heart rate were continuously recorded for 50 min. At the time of shock (t=0), 2.1 ml blood per 100 g body weight was taken at a rate of 1 ml/min (34-40% of total blood volume). At 45 min before induction of hemorrhagic shock, rats were subjected to vagotomy or sham vagotomy. In vagotomised animals a ventral cervical incision was made and both vagal trunks exposed. The vagus nerve was ligated at both ends using 4-0 silk suture and divided. In sham-operated animals, both vagal trunks were exposed, but the vagus nerve was not ligated and divided.

Before the experiments, 18 rats were fasted, 18 were fed with low-fat feed (6.9 en % protein, 75.4 en % carbohydrate, 16.7 en % fat) and 18 were fat with a high-fat feed according to the invention (6.9 en % protein, 40.9 en % carbohydrate, 52.2 en % fat). The low-fat and high-fat feeds were isocaloric. 3 ml was given at 18 h before hemorrhagic shock, and 0.75 ml at 2 h and 45 min before hemorrhagic by oral gavage. Fasted and high-fat fed rats underwent vagotomy or sham vagotomy. To investigate the role of CCK, six high-fat fed animas were injected i.v. with CCK-A (500 μg/kg) and CCK-B (500 μg/kg) receptor antagonists and six with control vehicle 25 min before induction of shock. Potential pro-inflammatory properties of both CCK-receptor antagonists were investigated by stimulation of peritoneal macrophages isolated from rats (3 rats) and injection of CCK-A and CCK-B receptor antagonists in rats not subjected to shock (3). Peripheral nicotinic receptors were blocked by intravenous administration of chlorisondamine at 25 min before induction of shock (6 rats) in high fat fed rats subjected to sham vagotomy, in order to determine whether the observed effect were specific for stimulation of the cholinergic anti-inflammatory pathway. Six fasted sham vagotomised rats treated with chlorisondamine were included as controls in order to account for the decrease in MAP (from 100 to 65 mm Hg) associated with administration of chlorisondamine. At 90 min after hemorrhagic shock, blood was taken and segments of small bowel were harvested for determination of gut permeability. Plasma was separated by centrifugation, frozen immediately and stored until analysis.

### Results:

It was found that the high-fat nutrition strongly reduced hemorrhagic shock induced TNF-α and IL-6 in rats that were subjected to sham vagotomy, compared with low-fat and fasted controls. Vagotomy abrogated the high-fat induced reduction in TNF-α (205±11 vs. 5±1 pg/ml [sham]) and IL-6 (80±5 vs. 19±9 pg/ml [sham]) after hemorrhagic shock compared to rats that underwent sham vagotomy.

Based on the reduction of circulating endotoxin levels, permeability of ileum segments for HRP and bacterial translocation to distant organs by high fat, and the reversion thereof by vagotomy, it was concluded that a parasympathetic neural control mechanism underlies the protective effect of enteral high-fat nutrition.

Levels of circulating triglycerides in rats treated with CCK-receptor antagonists were similar compared to control rats, indicating that lipid absorption in the acute phase is unaffected.

Administration of CCK-A and CCK-B receptor antagonists enhance plasma INF-α (251±30 pg/ml) and IL-6 (87±14 pg/ml) following hemorrhagic shock compared to controls (10 and 9 pg/ml, respectively). Furthermore, plasma endotoxin was elevated, HRP permeability was increased while more bacteria translocated to distant organs in rats treated with CCK-receptor antagonist. As stimulation of peritoneal macrophages did not trigger TNF-α release in receptor-treated rats and injection of antagonists in rats not subjected to hemorrhagic shock did not elicit TNF-α release and did not cause bacterial translocation or increased HRP leakage, it is concluded that the high-fat enteral nutrition inhibits the pro-inflammatory response and prevents loss of intestinal barrier integrity by way of activation of CCK receptors.

High-fat enteral nutrition enhanced circulating corticosterone after hemorrhagic shock in sham-vagotomised shock animals compared to fasted rats. However, both vagotomy compared to sham-vagotomised controls and administration of CCK-receptor antagonists did not significantly affect corticosterone levels in high-fm treated rats. This confirms that vagotomy does not affect corticosterone levels. Corticosterone increase may be caused by stress of oral gavage.

Administration of chlorisondamine did not cause additional hypotension or changes in heart rate before and just after induction of shock MAP was significantly lower during 50 min observation compared with vehicle-treated controls, but this did not affect the shock-induced inflammatory response and loss of gut barrier integrity. Chlorisondamine abrogated the inhibitory effect of high-fat enteral nutrition on circulating TNF-α. TNF-α and IL-6 levels in the high-fat rats treated with chlorisondamine were comparable to those of chlorisondamine-treated fasted rats. Inhibition of nicotinic receptors in high-fat rats by way of administration of chlorisondamine led to increased bacterial translocation to distant organs, increased permeability for HRP in ileum segments and elevated plasma endotoxin levels, compared to high-fat control rats.

These findings indicate that high-fat enteral nutrition inhibits inflammation by stimulation of nicotinic receptors by way of efferent vagal fibers.

The results show that high-fat enteral nutrition stimulates CCK-receptors centrally or peripherally by way of the afferent vagus nerve leading to inhibition of the inflammatory response by way of vagal efferents and nicotinic receptors. This provides a functional new mechanism for the interaction between nutrition and the immune response and has important implications. An undesired response to transient high amounts of dietary antigens, biological toxins and destructive endogenous lysozymes in the gut is prevented, got barrier function is preserved and homeostasis maintained.

Based on these findings, the fat nutrition of the invention will be therapeutic in various inflammatory disorders such as sepsis and inflammatory bowel disease characterised by an inflammatory response in which TNF-α is prominent and intestinal barrier function is impaired. This is particularly important with potentially lethal inflammatory responses after trauma or injury, which require quick intervention.

1-15. (canceled)

16. A method of attenuating an inflammatory response in a mammal comprising administering to the mammal a composition comprising a protein fraction and a lipid fraction, the lipid fraction comprising 8-50 wt. % of phospholipids.

17. The method according to claim 16, in which the lipid fraction comprises 10-35 wt % of phospholipids.

18. The method according to claim 17, in which the lipid fraction comprises 12-30 wt % of phospholipids.

19. The method according to claim 16, in which the lipid fraction further comprises 2-50 wt. % of mono- and diglycerides.

20. The method according to claim 16, in which the protein fraction comprises intact casein, intact whey protein, hydrolysed soy protein, or combinations thereof.

21. The method according to claim 16, in which the composition further comprises, based on the total weight of the composition when dry, 0.2 to 25 wt % betaine.

22. The method according to claim 16, in which the lipid fraction comprises between 42 and 90 en % of the composition.

23. The method according to claim 16, wherein the inflammatory response arises from the mammal having undergone surgery, radiotherapy, chemotherapy, acute (bacterial) infections, ulcerations, bacterial translocation, trauma, injury, allergen exposure, ischemic events, heart failure, burns or is likely to develop CARS, SIRS, ARF, sepsis, and (multiple) organ failure.

24. The method according to claim 16, in which the composition is administered between 5 min and 24 h after the inflammatory response.

25. The method according to claim 23, in which the composition is administered between 24 h and 5 min before surgery, transplantation, blood transfusion, radiotherapy, chemotherapy or allergen exposure.

26. The method according to claim 16 in which the administration of the composition stimulates vagal afferents of the parasympathetic nervous system centrally or peripherically via the gastrointestinal tract leading to attenuation of the inflammatory response via stimulation of nicotinic receptors by vagal efferents.

27. A method of mitigating or preventing acute inflammatory response in a mammal comprising administering to a mammal suffering from a chronic inflammatory disease or anticipating surgery, transplantation, blood transfusion, radiotherapy, chemotherapy or allergen exposure a nutritional composition comprising 42-90 en % of lipids, 6-50 en % of proteins and 0-50 en % of carbohydrates, wherein the administration is between 5 min and 24 h after onset of the acute inflammatory response.

28. A nutritional composition comprising:
(a) 10-50 en % of a protein fraction comprising at least 90 wt. % of one or more of casein, whey protein and hydrolysed soy protein; and
(b) 40-90 en % of a lipid fraction comprising 8-50 wt. % of phospholipids.

29. The nutritional composition according to claim 28, in which the lipid fraction comprises 10-35 wt % of phospholipids.

30. The nutritional composition according to claim 29, in which the lipid fraction comprises 12-30 wt % of phospholipids.

31. The nutritional composition according to claim 28, in which the lipid fraction further comprises 2-50 wt. % of mono- and diglycerides.

32. The nutritional composition according to claim 28, in which the composition further comprises, based on the total weight of the composition when dry, 0.2 to 25 wt % betaine.

33. A method of attenuating acute inflammatory response, comprising administering to a mammal suffering from or at risk of acute inflammatory response an effective amount of a protein composition, a lipid composition, or both, capable of stimulating the parasympathetic nervous system centrally or peripherically via the gastrointestinal tract thereby attenuating the acute inflammatory response via stimulation of nicotinic receptors by vagal efferents.
